(19) United States
(12) Patent Application Publication
(10) Pub. No.: US 2011/0105517 A1
(43) Pub. Date: May 5, 2011

(54) THERAPEUTIC AGENT FOR AMYOTROPHIC LATERAL SCLEROSIS

(76) Inventors: Joh-E Ikeda, Tokyo (JP); Hitoshi Osuga, Ibaraki (JP); Kazunori Tanaka, Kanagawa (JP)

(21) Appl. No.: 13/001,871

(22) PCT Filed: Jun. 16, 2009
(86) PCT No.: PCT/JP2009/060918

§ 371 Date: Dec. 29, 2010

(30) Foreign Application Priority Data
Jul. 7, 2008 (JP) 2008-177279

(51) Int. Cl. A61K 31/4985; A61P 25/28; A61P 25/00

(52) U.S. Cl. 514/250

(57) ABSTRACT

Disclosed is a safe and effective therapeutic agent that can alleviate the symptoms of the intractable disease ALS and slow the progress thereof. The therapeutic agent for amyotrophic lateral sclerosis contains bromocriptine or a pharmaceutically acceptable salt thereof as the active ingredient.

### TECHNICAL FIELD

The present invention relates to a therapeutic agent for amyotrophic lateral sclerosis containing bromocriptine or a pharmaceutically acceptable salt thereof as an active ingredient.

### BACKGROUND ART

Amyotrophic Lateral Sclerosis (ALS) is a progressive neurodegenerative disorder that almost exclusively affects upper and lower motor neurons. In this intractable disease, motor neurons in the spinal cord and in the brain degenerate and fall out, causing severe muscle atrophy, spasticity, etc., and ultimately leading to death from the paralysis of respiratory muscles within several years of the onset of the disease.

However, the etiology of ALS is still not fully understood and effective treatment methods have not been established. The only drug that has been approved for treating ALS currently is riluzole (brand name: Rilutek, Rhone-Poulenc-Rorer), however, its effectiveness is questioned by some, making the development of new treatment a high priority.

Currently, the therapeutic agents for ALS are being developed all over the world. One such approach in the development of new therapeutic agents focuses on the search based on the various hypotheses regarding the etiology of ALS. Major hypotheses include hyper excitation of neurons by excitatory amino acids, disorders in the immune system, oxidative stress and shortage of neurotrophic factors. Numerous approved drugs and compounds have been tested based on these hypotheses, however, no effective treatments have been found to date. For example, clinical trials on vitamin E and creatine have been carried out based on the oxidative stress hypothesis, but both attempts were terminated without success.

[Non-patent Cited Publication 1] Mitsumoto H., “Treatment Strategy for Amyotrophic Lateral Sclerosis (ALS)”, Brain and Nerve, April 2007, vol. 59, No. 4, pp. 383-391.

[Non-Patent Cited Publication 2] Traynor B. J., Bruijn L., Conwit R, Beal F, O'Neill G. et al, Neuroprotective agents for clinical trials in ALS: a systematic assessment, Neurology, 2006, vol. 67, pp. 20-27.

### DISCLOSURE OF THE INVENTION

### Problems to be Solved by the Invention

An object of the present invention is to provide a treatment method for the intractable disease, ALS. In particular, the present invention aims to provide a safe and effective therapeutic agent to alleviate the symptoms or to delay the progression of ALS.

### Means for Solving the Problems

The inventors of the present invention have been actively conducting research to identify an effective therapeutic agent for ALS, and have recently discovered that bromocriptine mesylate (brand name: Parlodel, Novartis Pharmaceuticals Corporation) which has been approved as a therapeutic agent for Parkinson's disease, exhibits a potent effect in delaying the progression of the symptoms of ALS.

How the inventors came to identify the effect of bromocriptine mesylate in alleviating the symptoms of ALS is discussed in detail below.

As mentioned above, oxidative stress is one of the suspected pathogenesis of ALS. The Inventors of the present invention therefore decided to search for a low molecular weight compounds that selectively inhibit oxidative stress-induced cell death, and decided to focus, as the target of the search, on the cell death inhibitory pathway involving Neuronal Apoptosis Inhibitory Protein (NAIP). NAIP was first identified as a factor involved in determining the severity of the Spinal Muscular Atrophy (SMA), a disease characterized by the degeneration and the loss of lower motor neurons. NAIP was also shown to be activated by the stimulus from hypoxia, inflammation, and oxidative stress. Its reported functions include selective and specific inhibition of oxidative stress-induced cell death, and inhibition of cell death of dopaminergic neurons in the Rat model of Parkinson's disease, among others. These findings suggest a role of NAIP in functioning as an endogenous protective factor against the neuronal cell death induced by the reactive oxygen species. The inventors of the present invention have focused on this point and developed a screening method for identifying compounds that activate the cell death inhibitory pathway involving NAIP. By screening the library containing known approved drugs and neurotropic compounds and by selecting those that exhibited strong inhibitory activity against oxidative stress-induced cell death, the inventors of the present invention successfully identified bromocriptine mesylate.

The efficacy of bromocriptine mesylate was further evaluated in vivo by utilizing the SOD1^{H46R} mouse, a mouse model for slow progression ALS developed by Dr. Itoyama and others at Tohoku University. In an administration test where the drug was given to ALS model mouse after the appearance of neurological symptoms, bromocriptine mesylate was found to be an effective treatment method. This invention came to completion based on these findings.

In summary, this invention provides a therapeutic agent for ALS containing bromocriptine or a pharmaceutically acceptable salt thereof as the active ingredient. A preferred aspect of the present invention is one in which the salt of bromocriptine is mesylate.

### Effects of the Invention

The therapeutic agent of this invention, when given to ALS patients, can alleviate the symptoms and delay its progression. In addition, the active ingredient contained in the therapeutic agent of this invention has already been approved as a drug and has been thoroughly tested for safety.

### BRIEF DESCRIPTION OF THE DRAWINGS

FIG. 1 is a graph showing inhibition of oxidative stress-induced cell death by bromocriptine mesylate in HeLa cells;

FIG. 2 is a graph showing inhibition of oxidative stress-induced cell death by bromocriptine mesylate in differentiated SH-SY5Y cell.

FIG. 3 is a graph (box plot) showing evaluation of motor function of ALS-SOD1^{H46R} mouse with or without bromocriptine mesylate treatment in the Vertical Climb Test.

FIG. 4 is a graph (Kaplan-Meier plot) showing the duration of survival after the onset of the disease in ALS-SOD1^{H46R} mouse with or without bromocriptine mesylate treatment.

### PREFERRED MODE FOR CARRYING OUT THE INVENTION

The therapeutic agent of the present invention for treatment of Amyotrophic Lateral Sclerosis (ALS) includes bromocriptine or a pharmaceutically acceptable salt thereof as an active ingredient.

Bromocriptine, an active ingredient used in the present invention, is one kind of free radical scavenger. As such, it is considered to have an inhibitory effect on oxidative stress-induced cell death.

Bromocriptine mesylate, the mesylate salt of bromocritptine, has been known to have therapeutic effects on such disorders as Parkinson's disease, acromegaly, gigantism, hyperprolactinemic pituitary adenoma, hyperprolactinemic anovulation, poor puerperal lactation, and galactorrhea.

Bromocriptine of this invention can be obtained using the methods described in U.S. Pat. Nos. 3,754,814 and 3,752,888 or similar methods, or can be obtained commercially in the form of bromocriptine mesylate (brand name: Parlodel, Novartis Pharmaceuticals Corporation).

Bromocriptine, the active agent used in this invention, can be used in an isolated form or in a pharmaceutically acceptable salt form. In the present invention, pharmaceutically acceptable salt means ordinary salt formed from appropriate, non-toxic, organic acids or inorganic acids that maintain the biological activity and characteristics of bromocriptine. Its acid addition salts can include, for example, those from inorganic acids such as hydrochloric acid, sulfuric acid, hydrogen bromide, phosphoric acid, hydroiodic acid, sulfamic acid and nitric acid, or those from organic acids such as mesylate, methanesulfonate, p-toluenesulfonic acid, acetic acid, oxalic acid, citric acid, succinic acid, malic acid, lactic acid and fumaric acid. Among these, mesylate is the most preferable since it can be readily obtained commercially as mentioned above.

Bromocriptine and the pharmaceutically acceptable salt thereof of the present invention include all isomers. For example, isomers due to the presence of chiral carbon (R/S, α/β, enantiomers and diastereomers), optical isomers having the optical activity to rotate the plane of polarized light (D/L, d/l), polarized bodies from chromatography (highly polarized, lowly polarized), or mixtures of the above in any combinations and racemic mixtures, are all included in the present invention.

The route of administration of the therapeutic agent of this invention is not restricted and can be administered orally or non-orally (intravenous, intramuscular, intradermal, subcutaneous injection, or nasal administration, etc.).

Examples of formulations that are appropriate for oral administration include tablets, pills, capsules, powders, granules, fine granules, solutions, and syrups, while formulations for non-oral administration include injections, infusions and suppositories.

As the therapeutic agent of the present invention, the active ingredient, bromocriptine, or its salt, can be administered directly to the patient, however, it is preferable to administer them as a formulation whose pharmaceutical composition comprises the active ingredient as well as pharmaceutically and pharmacologically acceptable additives. Pharmaceutically and pharmacologically acceptable additives include, for example, excipients, disintegrants, bonding materials, glidants (coating materials), food colors, diluents, bases, solvents, isotonizing agents, pH adjusting agents, stabilizing agents, aerosol propellants, adhesive materials, etc.

Excipients can include glucose, lactose, crystalline cellulose, D-mannitol, starch, calcium phosphate, etc.

Disintegrants can include, for example, the compounds listed above for excipients, as well as chemically modified starch, cellulose and the like, such as croscarmellose sodium, carboxymethyl starch sodium, and polyvinylpolypyrrolidone.

Binders can include, for example, hydroxypropylcellulose, hydroxypropylmethylcellulose, and gelatin as well as those compounds listed above for the excipients.

Glidants (coating material) can include, for example, ethyl cellulose, hydroxypropylcellulose, hydroxypropylmethylcellulose, shellac, talc, carnauba wax, paraffin, sucrose, polyethylene glycol, etc.

Solvents can include, for example, injectable distilled water, physiological saline, propylene glycol, etc.

Isotonizing agents can include, for example, glucose, sodium chloride, D-mannitol, glycerol, etc.

Stabilizing agents can include, for example, para-hydroxybenzoates such as methylparaben and propylparaben, alcohols such as chlorobutanol, benzyl alcohol, and phenylethyl alcohol, benzalkonium chloride, phenols such as phenol and cresol, thimerosal, dehydroacetic acid, sorbic acid, etc.

The therapeutic agent of the present invention can be administered to animals afflicted with ALS including humans but preferably to humans.

The therapeutic agent of the present invention can be used in combination with other medications. Combination with other medications can occur within the same pharmaceutical composition as the case with pharmaceutical adjuvants, but can also occur in other pharmaceutical compositions.

The meaning of the term treatment in this specification is used in the broadest sense, including all those that aim at delaying disease progression or those that aim at alleviating or reducing the symptoms.

The dose of the therapeutic agent of the present invention can be adjusted appropriately depending on the various conditions such as disease progression, severity of the symptoms, patient age, body weight, etc. For example, a preferable dose for an adult for a day would be around 1.25 to 22.5 mg, administered once or several times a day.

In addition, the active ingredient of the therapeutic agent of the present invention, bromocriptine mesylate, has already been clinically utilized for other purposes (for Parkinson's disease, etc., as noted above) and has no known toxicity problems.

### EXAMPLES

The present invention is further illustrated through the following examples, however, these examples in no way restrict the present invention.

In the examples, we examined the inhibitory activity of bromocriptine against cell death induced by oxidative stress using cell viability assay and investigated the efficacy of bromocriptine in vivo by using ALS-SOD1^{H46R} transgenic mouse.

In the examples, we used bromocriptine mesylate (TOCRIS Cookson Ltd., Cat. No: 0427) shown in formula (I) as the source of bromocriptine.

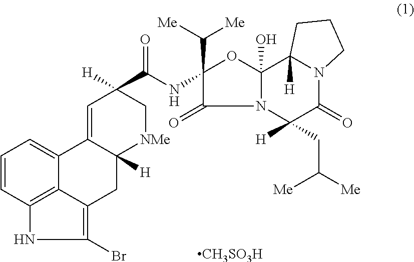

### Cell Lines:

HeLa (American Type Culture Collection, ATCC CCL2)

Human neuroblastoma SH-SY5Y (ATCC, CRL2266)

### Medium:

Cell Culture of HeLa cells: DMEM (Sigma) supplemented with 10% fetal bovine serum (FBS), 4 mM glutamate, 100 μg/ml streptomycin, 100 U/ml penicillin G.

Cell Culture of human neuroblastoma cell line SH-SY5Y:

DMEM (Sigma) supplemented with 10% fetal bovine serum (FBS), 100 μg/ml streptomycin, 100 U/ml penicillin G.

### Reagents:

Dimethylsulfoxide (DMSO: Sigma)

Menadione (Sigma)

AlamarBlue (Biosource international)

All-trans retinoic acid (Wako Pure Chemical Industries, Ltd.)

Carboxymethylcellulose (Carmellose sodium, CMC-Na) (Maruishi Pharmaceutical Co., Ltd.)

### Equipments:

Fluorescent plate reader (CYTOFLUOR™ Multi-Well Plate Reader Series 4000: PerSpective Biosystems)

### Example 1

### Cell Viability Assay 1

### Analysis of Oxidative Stress-Induced Cell Death in Non-Neuronal Cells

### (Methods)

HeLa cells (1.0×10⁶) were plated in 75 cm² flask and were cultured in the presence of 5% CO₂ at 37° C. for 16 hours. Bromocriptine mesylate in DMSO was then added to the medium at the final concentrations of 2.5, 5 and 10 μM. The amount of DMSO used in each samples were adjusted to that used in the 10 μM sample. Cells were cultured for additional 24 hours (bromocriptine mesylate treatment). As a negative control, DMSO without bromocriptine mesylate was added. Menadione was then added to the HeLa cells treated with bromocriptine mesylate at the final concentrations of 0, 20, 40, 60 and 80 μM and the cells were further incubated in 5% CO₂, at 37° C. (oxidative stress treatment). As a negative control, 0.1% Triton X-100/DMEM/10% FBS was added in place of menadione. After 4 hours of oxidative stress treatment, the medium was replaced with that containing AlamarBlue, and the cell culture was resumed in 5% CO₂, at 37° C. After 16 hours of incubation, the number of viable cells was counted by measuring the fluorescence at excitation wave length 530 nm/emission wave length 580 nm, using the fluorescence plate reader.

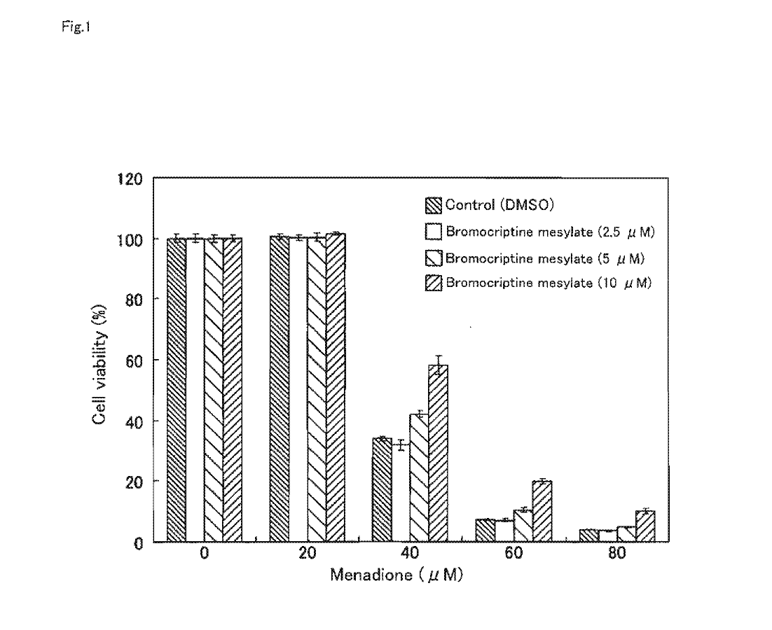

FIG. 1 shows the inhibitory effect of bromocriptine mesylate against cell death induced by menadione, an oxidative stress agent. The X-axis of the graph represents menadione concentration (μM), while the Y-axis of the graph represents cell viability (%). The Y-axis values under each condition are shown as viability relative to the no menadione treatment (0 μM) control which is set at 100%.

### (Results)

As shown in FIG. 1, the viabilities of HeLa cells pre-treated with bromocriptine increased depending on the concentration of bromocriptine mesylate in contrast to the cells pre-treated with DMSO alone and then exposed to the oxidative stress by the menadione. These results demonstrated that bromocriptine mesylate has an inhibitory effect against cell death induced by the oxidative stress.

### Example 2

### Cell Viability Assay 2

### Analysis of Oxidative Stress-Induced Cell Death in Differentiated Neuronal Cells

### (Methods)

SH-SY5Y cells were suspended in DMEM supplemented with 10% FBS, were plated at 1.0×10⁴ cells/well in 96 well microtiter plate, and were incubated in 5% CO₂ at 37° C. for 24 hours. The medium was then replaced with DMEM supplemented with 10% FBS and 10 μM all-trans retinoic acid (RA) (DMEM/FBS/RA), and after 5 days, bromocriptine mesylate in DMSO was added to the medium at the final concentrations of 5, 10, 20 and 40 μM. The amount of DMSO used in each samples were adjusted to that used in the 40 μM sample. The cells were cultured for additional 24 hours (bromocriptine mesylate treatment). As a negative control, DMSO without bromocriptine mesylate was added. Menadione was then added to the SH-SY5Y cells treated either with DMSO alone or bromocriptine mesylate at the final concentrations of 0, 20, 30, 40, and 50 μM, and cultured in 5% CO₂ at 37° C. (oxidative stress treatment). As a negative control, 0.1% Triton X-100/DMEM/10% FBS was added. After 4 hours of oxidative stress treatment, the medium was replaced with that containing AlamarBlue, and the cell culture was resumed in 5% CO₂, at 37° C. After 16 hours of incubation, the number of viable cells was counted by measuring the fluorescence at excitation wave length 530 nm/emission wave length 580 nm, using the fluorescence plate reader.

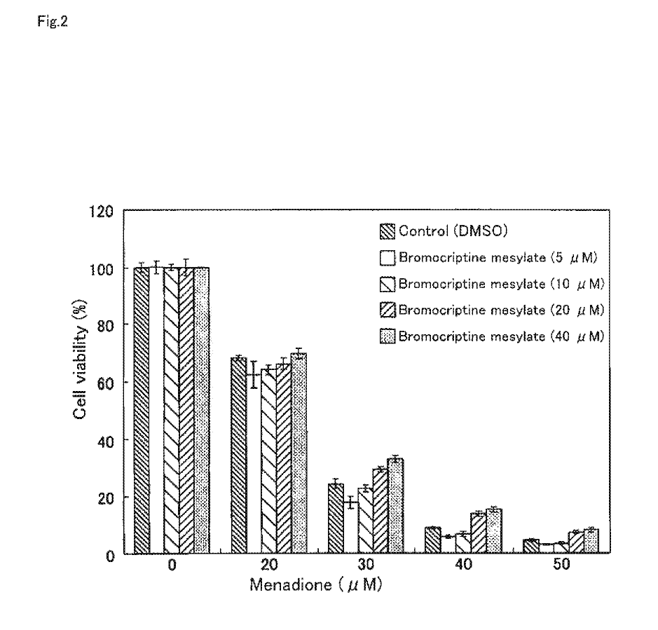

FIG. 2 shows the inhibitory effect of bromocriptine mesylate against the cell death induced by menadione, an oxidative stress agent. The X-axis of the graph represents menadione concentration (μM), while the Y-axis of the graph represents cell viability (%). The Y-axis values under each condition are shown as viability relative to the no menadione treatment (0 μM) control which is set at 100%.

### (Results)

As shown in FIG. 2, the viabilities of SH-SY5Y cells pre-treated with bromocriptine increased depending on the concentration of bromocriptine mesylate in contrast to the cells pre-treated with DMSO alone and then exposed to the oxidative stress by the menadione. These results demonstrated that bromocriptine mesylate has an inhibitory effect on cell death induced by the oxidative stress in differentiated SH-SY5Y cells.

### Example 3

### Efficacy Test In Vivo

### (Methods)

In this test, ALS-SOD1^{H46R} mouse carrying the mutation in the SOD1 gene (SOD1^{H46R}) (ALS1) was used. The mice were housed at 23° C. with a 12 hr light/dark cycle. Bromocriptine mesylate was suspended in 0.5% carboxymethylcellulose sodium (CMC-Na). The administration of bromocriptine mesylate was initiated when the neurological symptoms was first observed (first manifestation of the illness) in the ALS-SOD1^{H46R} mouse in the balance beam test. The dosage of bromocriptine mesylate was 10 mg or 1 mg per 1 kg of body weight in each of the animals, and administered by intraperitoneal injection once a day until the animals died (group treated with bromocriptine mesylate). As the negative control, the mice were administered with 0.5% CMC-Na (5 ml/kg) (group not treated with bromocriptine mesylate). Balance beam test (stainless beam: 50 cm long, 0.9 cm wide) was employed to evaluate the appearance of neurological symptoms. The evaluation of symptoms was carried out in 5 grades. Grade 3 was considered to be the first manifestation of the symptoms. The evaluation criteria used are shown below.

### <The Evaluation Criteria>

Grade 5: Crosses the beam normally, without any slipping of the hind legs.

Grade 4: Crosses the beam, with occasional slipping of the hind legs.

Grade 3: Crosses the beam barely, with frequent slipping of the hind legs.

Grade 2: Tries to cross the beam but falls.

Grade 1: Cannot stay on the beam.

The motor functions of mice in bromocriptine mesylate administered group and non-administered group were evaluated by vertical climb test (stainless bar: 50 cm long, 0.9 cm wide). The vertical climb tests were initiated when the mice reached 17 weeks of age. The vertical climb tests were carried out at a frequency of once a week, and repeated 5 times for each mouse. The best score among the 5 test results was recorded as the motor function value. Statistical analysis was carried out using SPSS 16.0.

### (Results)

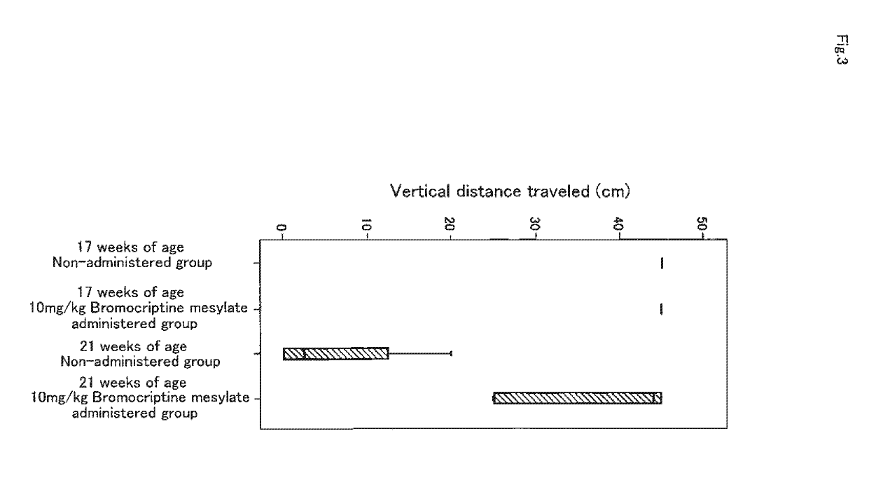

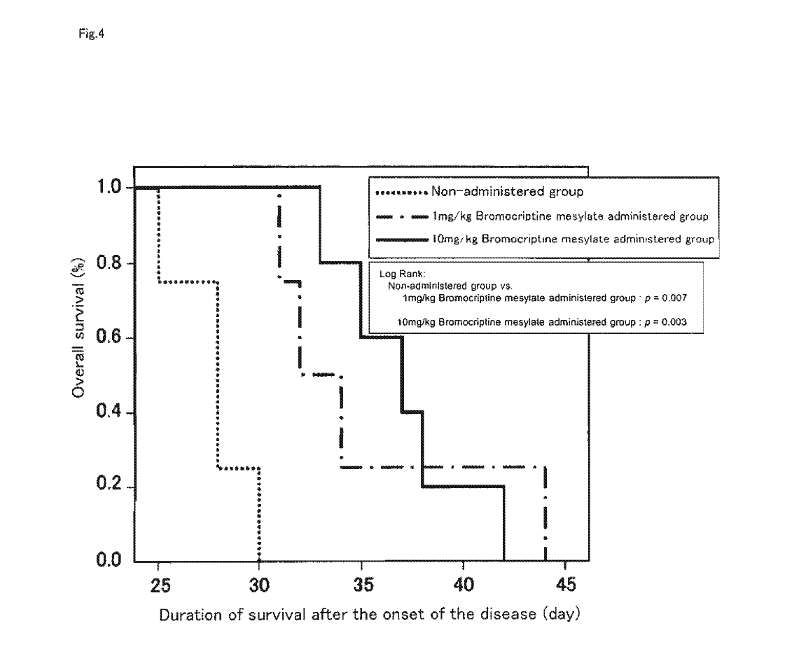

There was no difference in the motor function of ALS-SOD1^{H46R} transgenic mice in the bromocriptine mesylate administered group (10 mg/kg) (n=5) or in the non-administered group (n=4) at 17 weeks of age when the administration of the drug was initiated (see FIG. 3). In contrast, at 21 weeks of age, while the motor function of the bromocriptine mesylate administered group was largely unchanged, significant deterioration of motor function was apparent in the non-administered group (see FIG. 3). These results demonstrated that the quality of life (QOL) can be dramatically improved by the administration of bromocriptine mesylate. Overall survival after the onset of the disease (age at the onset of the disease: 125.9±2.8 days) was also significantly extended in bromocriptine mesylate administered groups in a dose dependent manner [35.3±6.0 day (n=4) (1 mg/ml) and 38.0±2.9 day (n=5) (10 mg/ml)] compared to the non-administered group [27.8±2.1 day (n=4)] (see FIG. 4).

1-2. (canceled)

3. A method of treating Amyotrophic Lateral Sclerosis by administering an effective dose of bromocriptine or a pharmaceutically acceptable salt thereof to a patient of Amyotrophic Lateral Sclerosis.

4. The method of treating Amyotrophic Lateral Sclerosis of claim 3 wherein the pharmaceutically acceptable salt of bromocriptine is a bromocriptine mesylate.
